(19) United States
(12) Patent Application Publication
(10) Pub. No.: US 2021/0035685 A1
(43) Pub. Date: Feb. 4, 2021

(54) REMOTE CONTROL FOR A MEDICAL APPARATUS, SYSTEM OF THE REMOTE CONTROL AND THE MEDICAL APPARATUS, AND METHOD FOR OPERATING THE MEDICAL APPARATUS

(71) Applicant: TRUMPF MEDIZIN SYSTEME GMBH + CO. KG, Saalfeld (DE)

(72) Inventor: Leonard ZEH, Saalfeld (DE)

(21) Appl. No.: 16/935,820

(22) Filed: Jul. 22, 2020

(30) Foreign Application Priority Data
Jul. 29, 2019 (EP) 19188807.2

(51) Int. Cl. G16H 40/67; A61G 13/02; G16H 20/40

(52) U.S. Cl. CPC G16H 40/67; A61G 2203/12; G16H 20/40; A61G 13/02

(57) ABSTRACT

A remote control for controlling a medical apparatus having at least one driven movable segment is provided. The remote control comprises a position sensor configured to detect a position and a motion of the remote control, and at least one release switch. The remote control is configured to emit a control signal capable of controlling a motion of the at least one driven movable segment according to a motion of the remote control detected by the position sensor when a control of the motion of the at least one driven movable segment is released by actuating the at least one release switch.

### CROSS-REFERENCE TO RELATED APPLICATIONS

This application claims the benefit of European Patent Application Serial No. 19188807.2, filed on Jul. 29, 2019, the entire disclosure of which is incorporated herein by reference.

### TECHNICAL FIELD

The present disclosure relates to a remote control for a medical apparatus, a system of the remote control and the medical apparatus, and a method for operating the medical apparatus. More specifically, the present disclosure is directed to a remote control for a medical apparatus having several movable segments driven in a controlled manner, a system of the remote control and the medical apparatus, and a method for operating the medical apparatus.

### BACKGROUND

A remote control for a medical apparatus which controls motions of movable segments of the medical apparatus, may, for security against unintended use, include at least one release switch and several switches for controlling motions in different directions of the respective movable segments. EP2455959A1 discloses such a remote control. However, in particular, in a case of a surgical operating table as the medical apparatus there are several movable segments. Therefore, the number of the switches actuated by buttons or soft keys of a touch screen increases and the existing remote controls have the disadvantage that, due to the increased number of buttons or soft keys, an operation of the surgical operating table, or other patient support apparatuses and medical devices, when a number of section are present, the operation becomes complicated. Additionally, an adjustment of a speed of the motion of the movable segments is not generally provided thereby further complicating teh operation of the surgical operating table.

### SUMMARY

The present disclosure includes one or more of the features recited in the appended claims and/or the following features which, alone or in any combination, may comprise patentable subject matter.

According to one aspect, the present disclosure provides an approach to overcome the above disadvantages and to provide a remote control for a medical apparatus enabling a comfortable operation of the medical apparatus in an intuitive manner.

According to another aspect of the invention, a remote control for controlling a medical apparatus having at least one driven movable segment comprises a position sensor configured to detect a position and a motion of the remote control, and at least one release switch. The remote control is configured to emit a control signal capable of controlling a motion of the at least one driven movable segment according to a motion of the remote control detected by the position sensor when a control of the motion of the at least one driven movable segment is released by actuating the at least one release switch.

In some embodiments, merely pressing of one button or one soft key for actuating the release switch is necessary for emitting a specific control signal since, after the release by the release switch, the control signal is then emitted due to the motion of the remote control itself.

In some embodiments of the remote control, the remote control is configured to emit the control signal according to a direction of the motion of the remote control detected by the position sensor with respect to a position of the remote control when the at least one release switch is actuated to start the release of the control of the motion of the at least one driven movable segment. When the control signal is emitted according to the direction of the motion of the remote control, an intuitive operation or motion of the movable segment is simply possible by moving the remote control according to a desired motion of the movable segment.

In other embodiments of the remote control, the remote control is configured to emit the control signal according to an amount of the motion of the remote control. By emitting the control signal according to the amount of the motion of the remote control, a more precise command including characteristics of the motion of the remote control can be emitted for controlling the medical apparatus easily in an intuitive manner.

In some embodiments of the remote control, the remote control is configured to emit the control signal according to a speed of the motion of the remote control. By emitting the control signal according to the speed of the motion of the remote control, also, a more precise command including characteristics of the motion can be emitted for comfortably controlling the medical apparatus in an intuitive manner.

In some embodiments of the remote control, the position sensor comprises an inclinometer. Inclinometers can be easily and economically obtained and, moreover, in some remote controls, an inclinometer is already included, e.g. for activating the remote control, so that the use for detecting the motion of the remote control for defining the control signal does not require further components of the remote control.

In some embodiments of the remote control, the position sensor comprises an acceleration sensor. Also acceleration sensors can be easily and economically obtained and, moreover, in some remote controls, an acceleration sensor is already included, e.g. for activating the remote control, so that the use for detecting the motion of the remote control for defining the control signal does not require further components of the remote control.

According to a further aspect of the invention, a system of a remote control and the medical apparatus is provided. The remote control is configured to control the motion of the at least one driven movable segment of the medical apparatus by the control signal as disclosed according to the prior aspects of the present disclosure. By the provision of this system, the medical apparatus can be comfortably operated without a need for searching an appropriate button or soft key for operating a specific motion of the movable segment of the medical apparatus.

In one embodiment of the system, the remote control is configured to control a direction of the motion of the at least one driven movable segment according to a direction of the motion of the remote control with respect to a position of the remote control when the at least one release switch starts to release the control of the motion. With this approach, the motion of the movable segment can be initiated intuitively so that the operation of the medical apparatus is facilitated.

In some embodiments of the system, the remote control is configured to control a speed of the motion of the at least one driven movable segment according to an amount of the motion of the remote control with respect to a position of the remote control when the at least one release switch starts to release the control of the motion, wherein the speed of the driven movable segment is the higher the larger the amount of the motion of the remote control is.

Therefore, a further characteristic of the motion of the movable segment can be determined in an easy and intuitive manner. Contrary to the existing remote controls, when possible anyway, it is not necessary to enter several menus to configure the speed of the motion.

In some embodiments of the system, the remote control is configured to control a speed of the motion of the at least one driven movable segment according to a speed of the motion of the remote control, wherein the speed of the motion of the at least one driven movable segment is the higher the higher the speed of the motion of the remote control is.

Therefore, a further characteristic of the motion of the movable segment can be alternatively or additionally determined in an easy and intuitive manner. Contrary to the existing remote controls, when possible anyway, it is not necessary to enter several menus to configure the speed of the motion.

In some embodiments of the system, the remote control has an elongated shape, the motion of the remote control detected by the position sensor is at least one of a tilting around an axis of the remote control perpendicular to the elongated shape and a tilting around a longitudinal axis of the remote control parallel to the elongated shape. In this embodiment, easy and unambiguous motions of the remote control are used for determining the motion of the movable segment of the medical apparatus so that the intuitive operation of the medical apparatus is easily possible.

In some embodiments of the system, the remote control is configured to stop the motion of the driven movable segment when the remote control is moved into a horizontal position. In such an embodiment, stopping of the motion of the movable segment is easily and intuitively possible.

In other embodiments of the system, the remote control comprises several release switches, the medical apparatus comprises at least one driven movable segment which performs several motions respectively driven by a driving device or the medical apparatus comprises several driven movable segments respectively driven by a driving device, and the several release switches are respectively assigned to a specific motion of one of the driven movable segments. In such a system, several movable segments of the medical apparatus can be controlled in an easy and intuitive manner. On the one hand, if one movable segment performs several motions, for example, by one release switch, a height adjustment of a tabletop of an operating table can be released and, by another release switch, tilting of the tabletop around its longitudinal axis can be released, therefore, the several release switches release the control of the motions of one movable segment. On the other hand, if merely one motion is executed by one of the driven movable segment, the control of this motion is released, so that the several release switches release the motions of several driven movable segments.

In some embodiments of the system, the remote control comprises a communication interface configured to establish a communication to the medical apparatus in a wireless manner. By the wireless communication between the remote control and the medical apparatus, a flexible use of the remote control without possibly disturbing medical personnel working at the medical apparatus is possible.

In another embodiment of the system, the remote control comprises a communication interface having a connection cable configured to establish a communication to the medical apparatus in a wired manner. By the wired communication, a safe and reliable communication without disturbances of obstacles or interference radiation is possible.

In some embodiments of the system, the medical apparatus is configured as a surgical operating table. In particular, an operating table can comprise a large quantity of movable segments for adjusting the position of a patient in a surgical treatment. Moreover, there is a necessity for adjusting the movable segments during surgery. Therefore, the easy and intuitive operation of the operating table is a tremendous advantage.

According to another aspect of the invention, a method for operating a medical apparatus by means of a remote control comprises the steps: releasing a control of a motion of a specific one of driven movable segments of the medical apparatus by actuating a specific release switch of the remote control and controlling a motion of the specific one of the movable segments by moving the remote control with respect to a position of the remote control when the specific release switch starts to release the control of the motion of the specific one of the movable segments.

In this way, the movable segment of the medical apparatus can be operated without a need for searching an appropriate button or soft key for operating a specific motion of the movable segment of the medical apparatus.

In an embodiment of the method, a direction of the motion of the specific one of the driven movable segments is defined by a direction of motion of the remote control around an axis perpendicular to an elongated shape of the remote control or around an axis parallel to the elongated shape of the remote control.

In some embodiments of the method, easy and unambiguous motions of the remote control are used for determining the motion of the movable segment of the medical apparatus so that the intuitive operation of the medical apparatus is easily possible.

In some embodiments of the method, the motion of the specific one of the driven movable segments is stopped when the remote control is moved into a horizontal position. In this way, stopping of the motion of the movable segment is easily and intuitively possible.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 shows a system of a remote control in a plan view and an operating table in a side view;

FIG. 2 shows a starting position and an end position of the remote control in a side view when the remote control is tilted around an axis perpendicular to an elongated shape of the remote control for controlling a motion;

FIG. 3 shows another starting position and the end position of the remote control in the side view when the remote control is tilted around the axis perpendicular to the elongated shape of the remote control for controlling a motion;

FIG. 4 shows a starting position and an end position of the remote control in a front view when the remote control is tilted around an axis parallel to an elongated shape of the remote control for controlling a motion;

FIG. 5 shows another starting position and the end position of the remote control in front view when the remote control is tilted around the axis parallel to the elongated shape of the remote control for controlling a motion; and

FIG. 6 shows a flow chart of a method according to the invention.

### DETAILED DESCRIPTION

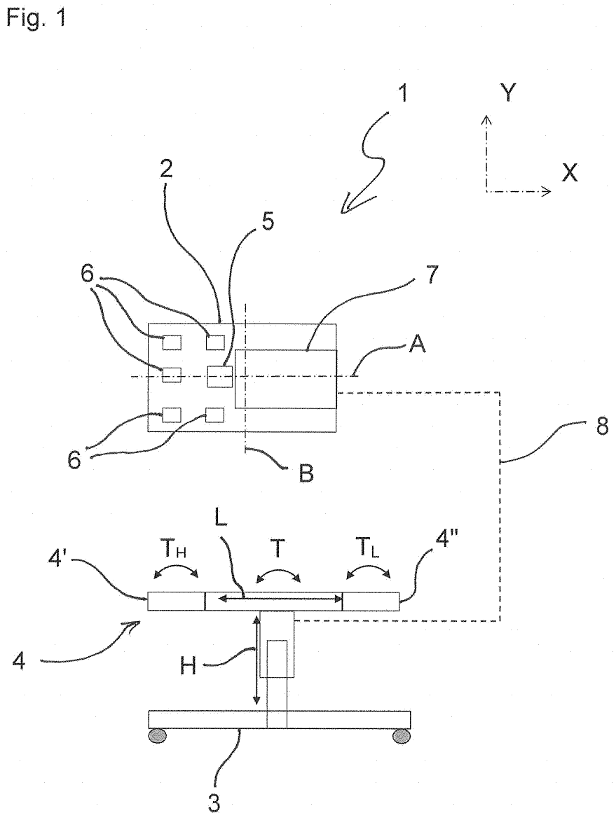

FIG. 1 shows a system 1 according to the present disclosure including a remote control 2 in a plan view and an operating table as an embodiment of a medical apparatus 3 in a side view.

The medical apparatus 3 comprises several movable segments, namely, an entire tabletop 4, and a head plate 4′ and a leg plate 4″ of the tabletop 4. The movable segments 4, 4′, 4″ are respectively driven by at least one driving device (not shown). The tabletop 4 is moved by a first driving device (not shown) in a vertical direction depicted by arrow H for adjusting a height of the tabletop 4. Further, the tabletop 4 is tilted by a second driving device (not shown) into a Trendelenburg position or an Anti-Trendelenburg position as depicted by arrow T. Furthermore, the tabletop 4 is displaced by a third driving device (not shown) along a longitudinal axis of the tabletop 4 as depicted by arrow L. The head plate 4′ is tilted by a fourth driving device (not shown) as depicted by arrow TH and the leg plate 4″ is tilted by a fifth driving device (not shown) as depicted by arrow TL. Alternatively, the medical apparatus 3 is provided with another number of driven segments 4, 4′, 4″, wherein at least one driven segment 4, 4′, 4″ movable in different directions is provided.

The remote control 2 comprises a position sensor 5. The position sensor 5 comprises an inclinometer. Alternatively or additionally, the position sensor 5 can comprise another type of sensor, e.g., an acceleration sensors or a gyroscope combined with a geomagnetic sensor. The position sensor 5 detects a position and a motion or an acceleration of the remote control 2.

Furthermore, the remote control 2 comprises five release switches 6. The release switches 6 are respectively assigned to a specific motion of one of the driven movable segments 4, 4′, 4″. By a first one of the release switches 6, the control of the vertical motion of the tabletop 4 can be released, by a second one of the release switches 6, the control of the tilting of the tabletop 4 into the Trendelenburg position or Anti-Trendelenburg position can be released. A third one of the release switches 6 releases the control of the displacement of the tabletop 4 in the longitudinal direction of the tabletop 4, a fourth one of the release switches 6 releases the control of the tilting of the head plate 4′, and a fifth one of the release switches 6 releases the control of the tilting of the leg plate 4″. In a further alternative embodiment, another number of release switches 6, e.g., according to a number of motions of the movable segments 4, 4′, 4″, merely one release switch 6, or more than two release switches 6, are provided.

The remote control 2 has an elongated shape and it has an axis A parallel to the elongated shape in a X axis direction of the remote control 2 and an axis B perpendicular to the elongated shape in a Y axis direction of the remote control 2. Alternatively, the remote control 2 does not have an elongated shape and the axes A and B, if necessary, are determined by definition.

The remote control 2 further comprises a communication interface 7. The communication interface 7 establishes a communication 8 to the medical apparatus 3 in a wireless manner. Alternatively, the communication interface 7 has a connection cable and establishes the communication 8 to the medical apparatus 3 in a wired manner.

By the communication 8, the remote control 2 emits a control signal capable of controlling the motion of the movable segments 4, 4′, 4″ according to a motion of the remote control 2 detected by the position sensor 5 when the control of the motion of the driven movable segments 4, 4′, 4″ is released by actuating the release switch 6. In particular, the remote control 2 emits the control signal according to a direction of the motion of the remote control 2 detected by the position sensor 5 with respect to a position of the remote control 2 when the release switch 6 starts to release the motion. The remote control 2 emits the control signal according to an amount of the motion of the remote control 2. Additionally, the remote control 2 emits the control signal according to a speed of the motion of the remote control 2. Alternatively, the amount and/or the speed of the motion of the remote control 2 are not considered, and, further alternatively, the direction of the motion is not considered but any motion is considered for moving the segment, e.g., in a reciprocating manner.

By emitting the control signal, the remote control 2 is configured to control the direction of the motion of the driven movable segments 4, 4′, 4″ according to the direction of the motion of the remote control 2 with respect to a position of the remote control 2 when the release button 6 starts to release the motion. Furthermore, the remote control 2 is configured to control a speed of the motion of the driven movable segments 4, 4′, 4″ according to the amount of the motion of the remote control 2 with respect to the position of the remote control 2 when the release button 6 starts to release the motion, wherein the speed of the driven movable segments 4, 4′, 4″ is higher the larger the amount of the motion of the remote control 2 is. Moreover, the remote control 2 is configured to control the speed of the motion of the driven movable segments 4, 4′, 4″ according to the speed of the motion of the remote control 2, wherein the speed of the motion of the driven movable segments 4, 4′, 4″ is higher the higher the speed of the motion of the remote control 2 is. In alternative embodiments, the amount and/or the speed of the motion of the remote control 2 is not considered for the control of the speed of the driven movable segments 4, 4′, 4″.

The motion of the remote control 2 detected by the position sensor 5 is a tilting around the axis B of the remote control 2 perpendicular to its elongated shape and a tilting around the longitudinal axis A of the remote control 2 parallel to its elongated shape. By the detection of the motion around the axis B of the remote control 2 perpendicular to its elongated shape, the vertical motion of the tabletop 4 and the displacement motions along the axis L are controlled. By tilting the remote control 2 in one direction, the tabletop 4 is lifted and, by tilting the remote control in the other direction, the tabletop 4 is lowered. By the detection of the motion around the axis A of the remote control 2 parallel to the elongated shape, the tilting motions of the table top 4, of the head plate 4′, and of the leg plate 4″ are controlled. In alternative embodiments, any of the motions are controlled either by detecting the motion around the axis A of the remote control 2 or by detecting the motion around the axis B of the remote control 2.

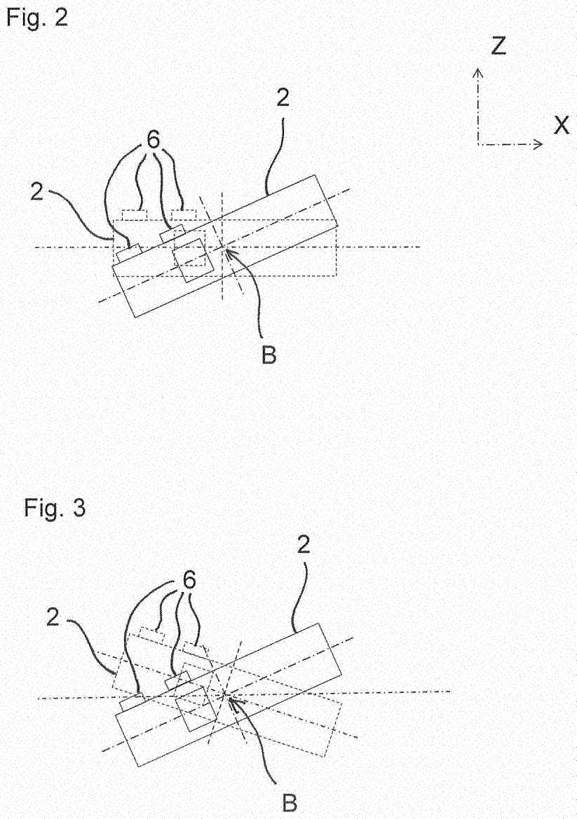

FIG. 2 shows a starting position and an end position of the remote control 2 in a side view when the remote control 2 is tilted around the axis B in the Y axis direction (FIG. 1) of the remote control 2 perpendicular to an elongated shape of the remote control 2 for controlling one of the motions. The initial position of the remote control 2 where the release switch 6 is actuated is depicted by dashed lines and the end position of the remote control 2 is depicted by solid lines. The speed and the direction of the motion of the driven movable segment 4, 4′, 4″, the control of which is released by actuating the respective release switch 6, are determined by the movement of the remote control 2 around the axis B. In FIG. 2, in the initial position of the remote control 2, a Z axis of the remote control 2 is oriented vertically so the remote control 2 is oriented in a horizontal position.

FIG. 3 shows another starting position and end position of the remote control 2 of FIG. 2 in the side view when the remote control 2 is tilted around the axis B perpendicular to the elongated shape of the remote control 2 for controlling one of the motions. The initial position of the remote control 2 where the release switch 6 is actuated is depicted by dashed lines and the end position of the remote control 2 is depicted by solid lines. This figure shows that the motion of the driven segment 4, 4′, 4″ of the medical apparatus 3 is not to be controlled compulsorily starting from the horizontal position of the remote control 2 but, also, another position is possible if the release switch 6 is actuated in this position and the remote control 2 is moved in a specific manner in order to control the motion.

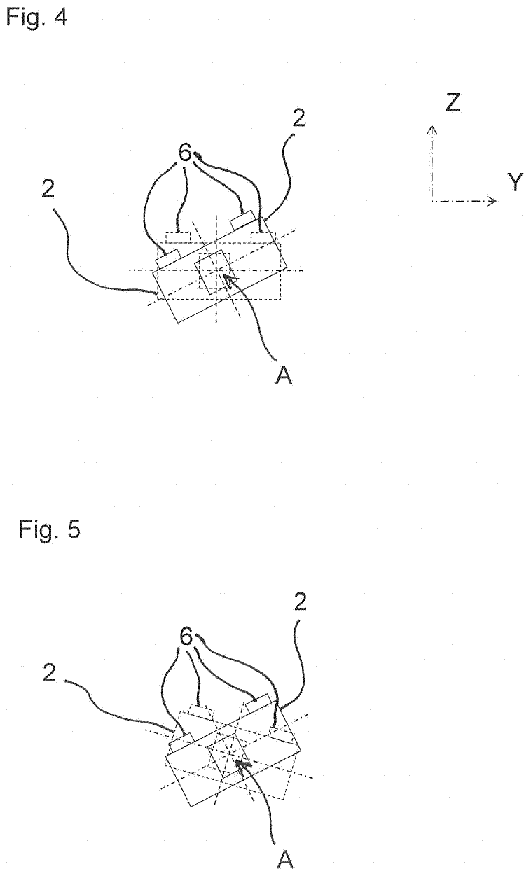

FIG. 4 shows the starting position and the end position of the remote control 2 in a front view when the remote control 2 is tilted around the axis A in the X axis direction (FIG. 1) of the remote control 2 parallel to an elongated shape of the remote control 2. The initial position of the remote control 2 where the release switch 6 is actuated is depicted by dashed lines and the end position of the remote control 2 is depicted by solid lines. The speed and the direction of the motion of the driven movable segment 4, 4′, 4″, the control of which is released by actuating the respective release switch 6, are determined by the movement of the remote control 2 around the axis A. In FIG. 4, in the initial position of the remote control 2, the Z axis of the remote control 2 is oriented vertically so the remote control 2 is oriented in a horizontal position.

FIG. 5 shows another starting position and end position of the remote control 2 of FIG. 4 in the front view when the remote control 2 is tilted around the axis A parallel to the elongated shape of the remote control 2 for controlling one of the motions. This figure shows that the motion of the driven segment 4, 4′, 4″ of the medical apparatus 3 is not to be controlled compulsorily starting from the horizontal position of the remote control 2 but, also, another position is possible if the release switch 6 is actuated in this position and the remote control 2 is moved in a specific manner in order to control the motion.

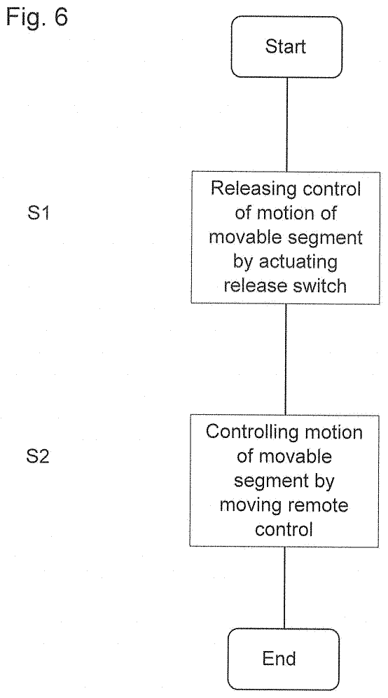

FIG. 6 shows a flow chart of a method according to the invention.

In use, in a first step S1, the control of a specific one of the motions of the driven movable segments 4, 4′, 4″ of the medical apparatus 3 is released by actuating a specific release switch 6 of the remote control 2 assigned to the specific motion.

In a next step S2, the motion of the specific one of the movable segments 4, 4′, 4″ is controlled by moving the remote control 2 with respect to a position of the remote control 2 when the specific release switch 6 is actuated.

The direction of the motion of the specific one of the driven movable segments 4, 4′, 4″ is defined by the direction of motion of the remote control 2 around the axis B perpendicular to an elongated shape of the remote control 2 or around the axis A parallel to the elongated shape of the remote control 2.

The remote control 2 emits a stop command for stopping the motion of the driven movable segment 4, 4′, 4″ when the remote control 2 is moved into a horizontal position so that the motion of the specific one of the driven movable segments 4, 4′, 4″ is stopped. This function is not compulsorily and, alternatively, the motion is stopped when the release switch 6 is not actuated anymore and the release switch 6 does not release the command anymore. The horizontal position is detected by the inclinometer or, alternatively, by the acceleration sensor. The acceleration sensor detects components of acceleration forces in the directions of three axes X, Y, Z of the remote control 2. If the component of the acceleration force in the direction of the Z axis of the remote control 2 amounts 1g, the remote control 2 is determined as being oriented in the horizontal position.

While embodiments have been illustrated and described in detail in the drawing and the foregoing description, such illustration and description are to be considered illustrative or exemplary and not restrictive. The invention is not limited to the disclosed embodiments. From reading the present disclosure, other modifications will be apparent to a person skilled in the art. Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. Although this disclosure refers to the specific embodiments, it will be understood by those skilled in the art that various changes in form and detail may be made without departing from the subject matter set forth in the accompanying claims.

1. A remote control for controlling a medical apparatus having at least one driven movable segment, the remote control comprising:
a position sensor configured to detect a position and a motion of the remote control, and
at least one release switch,
wherein the remote control is configured to emit a control signal capable of controlling a motion of the at least one driven movable segment according to a motion of the remote control detected by the position sensor when a control of the motion of the at least one driven movable segment is released by actuating the at least one release switch.

2. The remote control of claim 1, wherein the remote control is configured to emit the control signal according to a direction of the motion of the remote control detected by the position sensor with respect to a position of the remote control when the at least one release switch is actuated to start the release of the control of the motion of the at least one driven movable segment.

3. The remote control of claim 1, wherein the remote control is configured to emit the control signal according to an amount of the motion of the remote control.

4. The remote control of claim 1, wherein the remote control is configured to emit the control signal according to a speed of the motion of the remote control.

5. The remote control of claim 1, wherein the position sensor comprises an inclinometer.

6. The remote control of claim 1, wherein the position sensor comprises an acceleration sensor

7. A system comprising
a remote control including a position sensor configured to detect a position and a motion of the remote control, and at least one release switch, wherein the remote control is configured to emit a control signal capable of controlling a motion of the at least one driven movable segment according to a motion of the remote control detected by the position sensor when a control of the motion of the at least one driven movable segment is released by actuating the at least one release switch, and
a medical apparatus,
wherein the remote control is configured to control the motion of at least one driven movable segment of the medical apparatus by the control signal.

8. The system of claim 7, wherein the remote control is configured to control a direction of the motion of the at least one driven movable segment according to a direction of the motion of the remote control with respect to a position of the remote control when the at least one release switch starts to release the control of the motion.

9. The system of claim 7, wherein the remote control is configured to control a speed of the motion of the at least one driven movable segment according to an amount of the motion of the remote control with respect to a position of the remote control when the at least one release switch starts to release the control of the motion, wherein the speed of the at least one driven movable segment is the higher the larger the amount of the motion of the remote control is.

10. The system of claim 7, wherein the remote control is configured to control a speed of the motion of the at least one driven movable segment according to a speed of the motion of the remote control, wherein the speed of the motion of the at least one driven movable segment is as higher as higher the speed of the motion of the remote control is.

11. The system of claim 10, wherein the remote control is configured to emit a stop command for stopping the motion of the at least one driven movable segment when the remote control is moved into a horizontal position.

12. The system of claim 7, wherein
the remote control comprises several release switches,
the medical apparatus comprises at least one driven movable segment which performs several motions respectively driven by a driving device or the medical apparatus comprises several movable segments respectively driven by a driving device, and
the several release switches are respectively assigned to a specific motion of one of the driven movable segments.

13. The system of claim 7, wherein the remote control comprises a communication interface configured to establish a communication to the medical apparatus in a wireless manner.

14. The system of claim 7, wherein the remote control comprises a communication interface having a connection cable configured to establish a communication to the medical apparatus in a wired manner.

15. The system of claim 7, wherein the medical apparatus is configured as an operating table.

16. A method for operating a medical apparatus by means of a remote control with the steps:
releasing a control of a motion of a specific one of driven movable segments of the medical apparatus by actuating a specific release switch of the remote control, and
controlling a motion of the specific one of the movable segments by moving the remote control with respect to a position of the remote control when the specific release switch starts to release the control of the motion of the specific one of the movable segments.

17. The method of claim 16, wherein a direction of the motion of the specific one of the driven movable segments is defined by a direction of motion of the remote control around an axis (B) perpendicular to an elongated shape of the remote control or around an axis (A) parallel to the elongated shape of the remote control.

18. The method of claim 17, wherein the motion of the specific one of the driven movable segments is stopped when the remote control is moved into a horizontal position.
